(19) United States
(12) Patent Application Publication
(10) Pub. No.: US 2010/0143703 A1
(43) Pub. Date: Jun. 10, 2010

(54) ABRASION-RESISTANT FREE-FLOWING GLYCOCYAMINE-CONTAINING MOULDINGS AND METHODS FOR THEIR PRODUCTION

(76) Inventors: Stephan Winkler, Altenmarkt (DE); Roland Moller, Truchtlaching (DE); Susanne Erl, Peterskirchen (DE)

Correspondence Address: FULBRIGHT & JAWORSKI, LLP, 666 FIFTH AVE, NEW YORK, NY, 10103-3198, US

(21) Appl. No.: 12/452,710

(22) PCT Filed: Jul. 21, 2008
(86) PCT No.: PCT/EP2008/005958

§ 371 Date: Jan. 19, 2010

(30) Foreign Application Priority Data
Jul. 21, 2007 (DE) 10 2007 034 102.6

(51) Int. Cl. B32B 5/16; B29B 9/12

(52) U.S. Cl. 428/327; 264/13

(57) ABSTRACT

The invention relates to abrasion-resistant free-flowing glycocyamine-containing mouldings, in particular pellets and extrudates, and methods for their production. The bulk density of the mouldings is from 350 to 850 kg/m³, and their grain size range is from 32 to 2750 μm, and their glycocyamine content is from 55 to 99.9% by weight, based on the total weight, and they are particularly suitable as feed additives.

The present invention relates to free-flowing and abrasion-resistant glycocyamine-containing moldings and also methods for production thereof.

Glycocyamine is an endogenous substance in the bodies of vertebrates including in humans which occupies a central role in the biosynthesis of creatine. Creatine can not only be taken up via the diet, but also formed endogenously, wherein, as energy-rich phosphocreatine, it is an important energy reserve of muscles, in addition to adenosine triphosphate (ATP). In the resting state of the muscle, ATP can transfer a phosphate group to creatine, with phosphocreatine being formed which is then in direct equilibrium with ATP. During muscle work it is of critical importance to replenish the ATP stores as rapidly as possible. In the first seconds of maximum muscle loading, phosphocreatine is available. This can transfer a phosphate group to adenosine diphosphate in a very rapid reaction via the enzyme creatine kinase and thus reform ATP. This is also called the Lohmann reaction.

Beneficial effects of supplementation with creatine monohydrate have been known in humans for many years, especially in the field of sports nutrition, but also in the medical field. Beneficial effects of food supplementation have also been found in animals and creatine monohydrate was therefore recommended for use as feed additive and as meat and bonemeal substitute in animal nutrition. Since the ban on animal proteins in feeds from 2000 in the EU, many diets for breeding animals and fat stock have been changed over to purely vegetarian diets, with fishmeal also being substantially avoided, which is not included in the ban. The changeover to purely vegetarian diets led to losses in performance and, even after seven years, the purely vegetarian diets are inferior to those containing animal proteins. One reason for this inferiority is the lack of creatine. Earlier studies clearly showed that creatine monohydrate added to the feed can improve the growing performance when purely vegetarian diets are fed [Wallimann, T.; Pfirter, H. P.: Use of Creatine as a Feed Additive. EP1051914].

In addition to the undoubtedly beneficial effects, creatine monohydrate also has some disadvantages. The stability of this compound in aqueous solutions is highly restricted and creatine monohydrate, after oral intake, has only a low bioavailability. In addition, creatine monohydrate is a very expensive substance and the performance improvements achieved in the animal growth field are virtually compensated for by the costs. Most recently, therefore, glycocyamine has also been used as food supplement and feed which, compared with creatine, in aqueous solution has an astonishing stability and is significantly more bioavailable [Gastner, T.; Krimmer, H.-P.: Guanidinoacetic acid used as an animal food additive. EP1758463]. Glycocyamine is converted very efficiently and rapidly to creatine in the body. Therefore glycocyamine can be administered in significantly lower amounts than creatine for the same effect.

In the literature, a multiplicity of synthetic methods have already been described for producing glycocyamine. As early as 1861, Strecker carried out successfully for the first time the synthesis of glycocyamine from glycine and cyanamide [Strecker, M.; Jahresber. Fortschr. Chem. Verw., (1861), 530].

Similarly, Fromm in 1925 described the reaction of glycine hydrochloride with sodium cyanamide and hydrochloric acid to give glycocyamine hydrochloride [Fromm, E.; Justus Liebigs Ann. Chem., 442, (1925), 130-149].

Cyanamide was also used for producing glycocyamine by Vassel in two patents. It was reacted with glycine, wherein the pH was adjusted to 9.4 using sodium hydroxide [Vassel, B.; U.S. Pat. No. 2,654,779]. In addition, Vassel proposed the use of chloroacetic acid and ammonia. In this case, glycine hydrochloride was first formed. The resultant solution was subsequently adjusted to a pH>9 using sodium hydroxide solution and subsequently reacted with cyanamide [Vassel, B.; U.S. Pat. No. 2,620,354].

In 1903 the production of glycocyamine was described by Wheeler and Merriam by reacting glycine with S-methyl-isothiourea iodide in basic aqueous solution. In this case, potassium hydroxide was used as base [Wheeler, H. I.; Merriam, H. F.; Am. Chem. Journal, 29, (1903), 478-492.]. A very similar procedure was described by Fischl in 1934, wherein the base used was an excess of ammonia [Fischl, S.; (1931), U.S. Pat. No. 1,967,400].

In the known methods, glycocyamine generally occurs as finely crystalline white to slightly yellowish powder which has a considerable dust fraction (particles<63 μm). The median grain diameter can vary within wide ranges and is generally between 25 and 150 μm. The glycocyamine obtained from the known syntheses has a dust fraction of greater than 50% by weight and a free-flowing property of score 6 and therefore virtually cannot be used for industrial production of feeds.

The free-flowing property of powders, granules and extrudates can be determined, for example, via the flow behavior via test funnels having different outlet diameters (Feed Tech 9.10/2005, pages 23-26; see also the example section of the present invention). The free-flowing property in this case is rated with scores from 1 for a very good flow behavior to 6 for very poor flow behavior. For the production of industrial feeds, the solids used should achieve at least a score of 3.

For the abrasion resistance of feed granules and extrudates, to date no uniform test protocol has been established. A method which has proved to be reproducible and meaningful in practice is the abrasion (particles<63 μm) in an air jet sieve over a defined period at a defined subatmospheric pressure. In particular, measurement of the difference of the abrasion after 3 and 15 minutes at a subatmospheric pressure of 7200 pascals has established itself. In good granules this value is below 5% by weight. The method will be described in more detail in the example section.

The object of the present invention was therefore to provide free-flowing, abrasion-resistant and therefore low-dust products which are suitable, in particular, for incorporation into feeds, and also methods for production thereof.

This object is achieved according to the invention by providing free-flowing and abrasion-resistant glycocyamine-containing moldings having a bulk density between 350 and 850 kg/m³, a grain size spectrum of 32 to 2750 μm and a glycocyamine content of 55 to 99.9% by weight, based on the total weight.

It has been found that with the provision of the glycocyamine-containing moldings according to the invention the objective has been met in full, namely providing free-flowing, abrasion-resistant and therefore low-dust products for the feed industry which are distinguished by good handling quality.

According to a preferred embodiment, these are granules and extrudates having a bulk density between 400 and 800 kg/m³ and, in particular, between 450 and 750 kg/m³. In addition, it is considered preferable that the grain size spectrum of the claimed moldings is between 32 and 1000 μm, and preferably less than 10% by weight of the particles are less than 100 μm and less than 10% by weight of the particles are above 850 μm. The moldings have a glycocyamine content of preferably 85 to 99% by weight, in particular 95 to 98.5% by weight.

In a preferred embodiment the moldings contain organic or inorganic binders in amounts of 0.05 to 15% by weight, preferably 0.1 to 1.5% by weight, which are suitable for use of the products according to the invention as feed additives. Preferably, the binders used for the production of moldings are byproducts or starting substances from the production process of glycocyamine such as glycine or salts of glycocyamine, and so these do not have to be separated off in advance for purifying the product. This proved to be particularly advantageous if small amounts of these substances still adhere in dissolved form to the glycocyamine used, wherein this can also be glycocyamine itself dissolved in water.

Especially suitable is also the addition of other binders such as, for instance, methylcellulose, ethylcellulose, carboxymethylcellulose, carboxyethylcellulose, carboxypropylcellulose, hydroxypropylmethylcellulose, hydroxymethylcellulose, microcrystalline cellulose, ethylmethylcellulose and other cellulose derivatives, starch, hydroxypropyl starch, native starch, pregelatinized or modified starch, sugar, sugar syrup, dextrin, gelatin, propyl vinyl alcohol, polyvinyl pyrrolidone, xanthan, glycocyamine salts, gum arabic, sodium chloride, sodium carbonate, sodium hydrogencarbonate and glycerol and also mixtures thereof.

For improvement of the free-flowing property of the moldings, it can be advantageous that said moldings contain a flow enhancer, in particular a hydrophilic and/or a hydrophobic silicic acid and/or silicate-based additives and/or fatty acids and/or salts thereof, such as stearic acid or palmitic acid and also sodium, potassium, and calcium salts thereof. The flow enhancers and binders are added to the glycocyamine in dry form, as suspension, or solution, before shaping, wherein amounts of 0.01 to 5% by weight have proved suitable.

The present invention further provides that the glycocyamine-containing moldings can optionally contain up to 40% by weight, in particular 1 to 10% by weight, of another nutritionally active substance from the group carbohydrates, fats, amino acids, proteins, vitamins, minerals, trace elements, and also derivatives thereof and mixtures thereof. Those which are preferred are considered to be, in particular, the essential amino acids lysine, threonine, methionine and tryptophan, and in addition vitamin A, vitamin D3, vitamin E, nicotinic acid, nicotinamide, β-carotene, fishmeal and casein.

The glycocyamine-containing moldings according to the invention should preferably have a free-flowing property of score 3, particularly preferably of score 2 or 1, and also an abrasion resistance of less than 12% by weight, preferably less than 10% by weight, and particularly preferably less than 4% by weight.

The invention further relates to a method for producing glycocyamine-containing moldings, characterized in that said moldings are obtained by shaping, in particular mixed granulation or shaping extrusion of a composition of glycocyamine and water and subsequent drying. The methods according to the invention can be carried out either continuously or as batch processes.

Extruders have proved to be particularly suitable for the shaping, in particular single-screw extruders, twin-screw extruders, ring-die presses and grinders. The solid used in this case is forced through an extrusion die generally at pressures up to 80 bar and temperatures of 20 to 120° C. The extrudate size can either be set by mechanical slicing or decomposition of the extrudates occurs with suitable selection of the processing parameters. By this means extrudates between 32 and 2750 μm, in particular between 32 and 1000 μm, can be generated.

In addition, granulators have proved to be particularly suitable for the shaping, in particular intensive mixers, vertical granulators, spray granulators, ring-layer granulators and plowshare mixers. The solid used is exposed in this case to high shear forces wherein, depending on the type, size and capacity of the granulator, velocities of 300 to 2500 revolutions per minute have proved to be suitable. The granulation can be carried out at temperatures between 20 and 120° C., wherein the method described delivers granules between 32 and 2750 μm, in particular between 32 and 1000 μm.

Advantageously, the glycocyamine used for producing the moldings is produced from glycine and cyanamide in an aqueous solvent, in particular water, with addition of a base. Methods of this type are described, for example, in U.S. Pat. No. 2,654,779 and U.S. Pat. No. 2,620,354. In a preferred embodiment, a water-moist glycocyamine directly from the production process is used for the granulation and extrusion, to which dissolved glycocyamine and/or starting materials or byproducts from the production process still adhere. A residual moisture of 15 to 25% by weight of the material used has proved to be particularly advantageous in this case. Overall, the mixtures used for the shaping can contain between 40 and 93% by weight of glycocyamine, between 7 and 60% by weight of water, between 0 and 15% by weight of binder and also 0 to 40% by weight of another nutritionally active substance.

It has proved to be particularly advantageous not only for the granulation but also for the extrusion if the glycocyamine used has a median grain size diameter of <95 μm, preferably <25 μm, and in particular <15 μm. In addition, amorphous glycocyamine, especially, has proved to be particularly suitable. For the invention, it is therefore considered to be preferred that more than 40%, preferably more than 90% of the material used is in the amorphous form. Particularly suitable amorphous glycocyamine for the granulation may be generated, firstly by setting suitable process parameters in the production process which are known to those skilled in the art, and also by milling crystalline material.

In order to obtain stable dry granules or extrudate, the moldings are dried. In this case, especially moving-bed driers and fluidized-bed driers have proved to be particularly gentle in order to avoid mechanical destruction of the still moist moldings. Preferably, temperatures between 50 and 130° C. and optionally vacuum are used.

The dust content of the resultant granules and extrudates is less than 5% by weight, preferably less than 2% by weight, measured by the method of Dr. Groschopp.

Owing to the very good abrasion resistance and free-flowing property, the glycocyamine-containing moldings according to the invention are outstandingly suitable as feed additive.

The examples hereinafter are intended to illustrate the present invention in more detail.

### EXAMPLES

### 1. Methods for Determining Abrasion Resistance and Free-Flowing Property

### 1.1 Abrasion Resistance

The test means consist of
air jet sieve
analytical sieve 63 μm
analytical balance (accuracy 0.01 g)
industrial vacuum cleaner
weighing dish

25 g of the granules or extrudate to be determined are weighed out and sieved for 3 min at 7200 pascal subatmospheric pressure on the air jet sieve and subsequently reweighed. The difference (=AW1) is equal to the abrasion resistance after 3 min. Subsequently this operation is repeated with the same sample and at the same settings for 12 min sieving time and the sample reweighed. The difference from the original weight (=AW2) is equal to the abrasion resistance after 15 min.

$${R}_{x} = \frac{( EW - {AW}_{x} ) * 100}{EW}$$

$$R = Abrasion resistance [ \% ]$$

$$AW = Weight after sieving [ g ]$$

$$EW = Initial weight [ g ]$$

The difference between the values at 3 min and 15 min is a measure of the abrasion resistance. The higher this value, the more abraded material is generated.

### 1.2 Free-Flowing Property

The test means consist of five test funnels having the same diameter and angle of inclination, but having different outlet diameters (2.5 mm; 5 mm; 8 mm; 12 mm and 18 mm). The solid to be determined is charged for this purpose into the test funnel, with the outlet being closed from the bottom in order that no material can run out during charging. In the next step the outlet is opened completely, without shaking the test funnel, and so the complete outlet cross section is cleared. The assessment parameter is the diameter at which the solid trickles through spontaneously and without external action. In this test the following apply:
Solid trickles through the 2.5 mm outlet: score 1
Solid trickles through the 5 mm outlet: score 2
Solid trickles through the 8 mm outlet: score 3
Solid trickles through the 12 mm outlet: score 4
Solid trickles through the 18 mm outlet: score 5
Solid does not trickle through the 18 mm outlet: score 6

### 2. Production of Granules and Extrudates

2.1 In a 75 liter intensive mixer (from Eirich), 34 kg of glycocyamine (KGA×50 value (median grain size diameter)=13.6 μm) having a water content of 20.8% were charged at room temperature and homogenized for 1 min. Subsequently, 269 g of starch are added under slow stirring. Thereafter the mixer contents are stirred at 1500 rpm, in the course of which the temperature increased to approximately 50° C. After 5 min of granulation time, granules of the desired grain size range were obtained. The resultant glycocyamine granules were dried in a fluidized-bed drier to a product temperature of 80° C. and subsequently the coarse fraction above 1.00 mm was sieved off.
2.2 In a vertical granulator, 35 kg of glycocyamine (KGA×50 value=23.2 μm) having a residual moisture of 13% were charged at room temperature and homogenized for 1 min. Subsequently, 305 g of starch and 2.60 kg of water were added with slow stirring. Thereafter the mixer contents were stirred at 2000 rpm, in the course of which the temperature increased. After 8 min of granulation time, granules in the desired grain size range were obtained. The granules were dried in the vacuum drying cabinet at 80° C. and 50 mbar.
2.3 4.3 kg of glycocyamine (KGA×50 value=12.6 μm) having a residual moisture of 20.7% were charged in a mixer and 34 g of starch were mixed in with slow stirring. Subsequently the mixture was placed in a ring-die extruder and forced through a die having 0.7 mm bore holes. The resultant extrudate was dried on a fluidized-bed drier to a product temperature of 50° C.
2.4 In a 75 liter intensive mixer (from Eirich), 34 kg of glycocyamine (KGA×50 value=63.8 μm) with a residual moisture of 9.4% were charged at room temperature and homogenized for 1 min. Subsequently, 308 g of starch and 3.57 kg of water were added with slow stirring. Thereafter the mixer contents were stirred at 1500 rpm, in the course of which the temperature increased to approximately 50° C. After 6 min of granulation time, a further 0.94 kg of water were added and the mixture was again granulated for a further 6 min. The resultant granules were dried in the vacuum drying cabinet at 80° C. and 50 mbar.

TABLE 1
Sieving analysis, free-flowing property,
abrasion resistance and bulk density of the
resultant granules

| | | Example 2.1 | Example 2.2 | Example 2.3 | Example 2.4 |
|---|---|---|---|---|---|
| Sieving analysis [%] | <63 μm | 1.3 | 4.4 | 1.4 | 8.6 |
| | 63-100 μm | 2.8 | 0.4 | 0.8 | 12.6 |
| | 100-200 μm | 7.8 | 10.8 | 0.6 | 30.1 |
| | 200-315 μm | 17.2 | 9.5 | 0.8 | 22.3 |
| | 315-500 μm | 47.5 | 14.5 | 2.3 | 24.7 |
| | 500-710 μm | 19.0 | 30.5 | 89.8 | 1.6 |
| | 710-850 μm | 3.3 | 21.5 | 3.6 | 0.0 |
| | ^{>850 μm} | 1.2 | 8.4 | 0.7 | 0.0 |
| Free-flowing property [score] | | 2 | 2 | 3 | 3 |
| Fraction < 63 μm [%] | | 1.3 | 4.4 | 1.4 | 8.6 |
| Bulk density [g/l] | | 587 | 617 | 532 | 426 |
| Abraded | after 3 min | 1.3 | 7.9 | 1.6 | 8.6 |
| material < 63 μm [%] | after 15 min | 4.4 | 14.8 | 4.8 | 17.0 |
| | difference between 3 | 3.1 | 6.9 | 3.2 | 8.4 |
| | and 15 min | | | | |

1-21. (canceled)

22. An abrasion-resistant and free-flowing glycocyamine-containing molding having a bulk density of from 350 to 850 kg/m³, a grain size spectrum of from 32 to 2750 μm and a glycocyamine content of from 55 to 99.9% by weight, based on the total weight of said molding.

23. The molding of claim 22, wherein a said molding contains binder in an amount of from 0.05 to 15% by weight.

24. The molding of claim 23, containing from 0.1% to 1.5% binder by weight.

25. The molding of claim 22, wherein said binder comprises a cellulose derivative.

26. The molding of claim 23, wherein said binder comprises methylcellulose, ethylcellulose, carboxymethylcellulose, carboxyethylcellulose, carboxypropylcellular, hydroxypropylmethylcellular, hydroxymethylcellulose, microcrystalline cellulose, ethylmethylcellulose starch, hydroxypropyl starch, native starch, pregelatinized or modified starch, sugar, sugar syrup, dextrin, gelatin, propyl vinyl alcohol, polyvinyl pyrrolidone, xanthan, glycine, a glycocyamine salt, gum arabic, sodium chloride, sodium carbonate, sodium hydrogen carbonate, glycerol and mixtures thereof.

27. The molding of claim 22, wherein molding has a grain size spectrum of 32 μm to 1000 μm.

28. The molding of claim 27, wherein less than 10% by weight of said particles are below 100 μm by weight, and less than 10% by weight of said particles are above 850 μm.

29. The molding of claim 22, wherein said molding has a glycocyamine content of 85 to 99% by weight.

30. The molding of claim 29, having a glycocyamine content of 95% to 98.5% by weight.

31. The molding of claim 22, wherein said molding has a bulk density between 400 to 800 kg/m³.

32. The molding of claim 31, having a bulk density of between 450 and 750 kg/m³.

33. The molding of claim 22, wherein said molding contains up to 40% by weight of other nutritionally active substances selected from the group of carbohydrates, fats, amino acids, proteins, vitamins, minerals, trace elements, and derivatives thereof.

34. The molding of claim 22, wherein said molding has a free-flowing property of score 3.

35. The molding of claim 22, having a free-flowing property of score 2 or 1.

36. The molding of claim 22, wherein said molding has an abrasion resistance of less than 12% by weight.

37. The molding of claim 14, having an abrasion resistance less than 10% by weight.

38. The molding of claim 37, having an abrasion resistance less than 4% by weight.

39. The molding of claim 22, wherein said molding contains a hydrophilic or hydrophobic silicic acid, a silicate-based additive, a fatty acid, a salt thereof or mixtures thereof in an amount of from 0.01 to 5% by weight.

40. A method for producing the molding of claim 22, comprising mixed granulation of a composition of glycocyamine and water followed by drying of granules which result therefrom.

41. The method of claim 49, comprising granulating with a intensive mixer a vertical granulator, a spray granulator, a ring-layer granulator, or a ploughshare mixer.

42. A method for producing the molding of claim 22, comprising shape extrudate or a composition of glycocyamine and water followed by drying of extrudates.

43. The method of claim 41, comprising extruding with a single-screw extruder, a twin-screw extruder, a ring-die press, or a grinder.

44. The method of claim 40 or 42, comprising using water-moist glycocyamine separated off from the production process and having a residual moisture of 15 to 25%.

45. The method of claim 40 or 42, wherein the mixture contains from 40 and 93% by weight of glycocyamine, from 7 to 60% by weight of water, up to 15% by weight of binder, up to 40% by weight of another nutritionally active substance.

46. The method of claim 40 or 42, wherein said glycocyamine has a median grain size diameter of <95 μm.

47. The method of claim 46, wherein said glycocyamine has a median grain size diameter of <25 μm.

48. The method of claim 46, wherein said glycocyamine has a median grain size diameter of <15 μm.

49. The method of claim 40 or 42, wherein more than 40% by weight of said glycocyamine is in amorphous form.

50. The method of claim 40 or 42, further comprising drying said molding optionally under vacuum at a temperature of from 50 to 130° C.

51. The method of claim 48, comprising drying said molding under a vacuum.

52. The method of claim 40 or 42, wherein said glycocyamine is produced from reaction of glycine and cyanamide in an aqueous solvent, with addition of a base.
